(19) United States
(12) Patent Application Publication
(10) Pub. No.: US 2026/0120277 A1
(43) Pub. Date: Apr. 30, 2026

(54) SYSTEMS AND METHODS FOR TUMOR MARGIN MEASUREMENT AUTOMATION

(71) Applicant: Mars, Incorporated, McLean, VA (US)

(72) Inventors: Andrew John BURICH, Chadds Ford, PA (US); Fernando Rodrigues JUNIOR, São José dos Campos (BR); Mark Justin PARKINSON, Long Valley, NJ (US); Jodie GERDIN, Ithaca, NY (US); Wilson YAU, Fountain Valley, CA (US)

(21) Appl. No.: 19/352,898

(22) Filed: Oct. 8, 2025

Related U.S. Application Data
(60) Provisional application No. 63/712,054, filed on Oct. 25, 2024.

(51) Int. Cl. G06T 7/00; G06T 7/11; G16H 70/60

(52) U.S. Cl. CPC G06T 7/0012; G06T 7/11; G16H 70/60; G06T 2207/20081; G06T 2207/20084; G06T 2207/30088; G06T 2207/30096

(57) ABSTRACT

A method for automating margin measurement for mass removal that utilizes one or more machine learning models is disclosed. The one or more machine learning models may assist in identifying and segmenting each biopsy in an image, classifying the biopsy as a clear biopsy or an ambiguous biopsy, rotating the image of the biopsy to a proper orientation, and identifying and segmenting the skin and tumor found in the image of the biopsy. The method may then determine a lateral and deep margin value and display the values on the image.

### CROSS-REFERENCE TO RELATED APPLICATION(S)

This patent application claims the benefit of priority to U.S. Provisional Application No. 63/712,054, filed on Oct. 25, 2024, the entirety of which is incorporated herein by reference.

### TECHNICAL FIELD

This present disclosure relates generally to the field of pathology and medical technology. In particular, the present disclosure relates to employing machine learning (ML) methodologies for analyzing images of biopsies to generate relevant measurements.

### BACKGROUND

Histopathologic examination is an important test for the diagnosis of many diseases in modern healthcare, and it plays a particularly critical role in cancer care. When a biopsy is performed to remove a mass (e.g., tumor) associated with the skin, pathologists take certain measurements to determine if the mass has been completely excised. As an example, such measurements quantify the distance between healthy tissue and the mass. Obtaining these measurements is a time consuming, unstandardized, and labor-intensive process. Thus, there is a need to standardize and automate this process.

### SUMMARY OF THE DISCLOSURE

The present disclosure solves the technical challenges typically encountered during the use of a conventional method, such as those discussed above. Specifically, the present disclosure solves the technical challenges and shortcomings by utilizing a unique combination of machine-learning models as well as rule-based algorithm(s) to make biopsy-related measurements and predict disease recurrence risk.

In some aspects, the techniques described herein relate to a computer-implemented method for automating margin measurement for mass removal including: receiving, by one or more processors, an image including one or more biopsies from a database; inputting the image, by the one or more processors, into a biopsy segmentation machine learning model; identifying and segmenting, by the one or more processors, based on an application of the biopsy segmentation machine learning model, a biopsy of the one or more biopsies in the image to form a base image including the biopsy, wherein the biopsy includes a mass and surrounding tissue; inputting the base image, by the one more processors, into an ambiguity classifier machine learning model; determining, by the one or more processors, based on an application of the ambiguity classifier machine learning model, the mass in the base image is distinct from the surrounding tissue; classifying, by the one or more processors, based on the application of the ambiguity classifier machine learning model, the base image as a valid image; inputting the valid image, by the one or more processors, into a rotation classifier machine learning model; determining, by the one or more processors, based on an application of the rotation classifier machine learning model, a rotation angle associated with the valid image; rotating, by the one or more processors, the valid image based on the rotation angle to form a properly oriented image, such that skin of the biopsy is at a top of the valid image; inputting the properly oriented image, by the one or more processors, into a skin and tumor segmentation machine learning model; identifying and segmenting, by the one or more processors, based on an application of the skin and tumor segmentation machine learning model, the skin and the mass in the properly oriented image to form a marked image; determining, by the one or more processors, a lateral margin value and a deep margin value based on the segmented skin and the mass shown in the marked image; and generating, by the one or more processors, computer-executable instructions configured to cause a user computing device to construct and display a graphical user interface (GUI) that presents the lateral margin value and the deep margin value in association with the marked image.

In some aspects, the techniques described herein relate to a system including: one or more processors of a computing system; and at least one non-transitory computer-readable medium storing instructions which, when executed by the one or more processors, cause the one or more processors to: receive an image including one or more biopsies from a database; input the image into a biopsy segmentation machine learning model; identify and segment, based on an application of the biopsy segmentation machine learning model, a biopsy of the one or more biopsies in the image to form a base image including the biopsy, wherein the biopsy includes a mass and surrounding tissue; input the base image into an ambiguity classifier machine learning model; determine, based on an application of the ambiguity classifier machine learning model, the mass in the base image is distinct from the surrounding tissue; classify, based on the application of the ambiguity classifier machine learning model, the base image as a valid image; input the valid image into a rotation classifier machine learning model; determine, based on an application of the rotation classifier machine learning model, a rotation angle associated with the valid image; rotate the valid image based on the rotation angle to form a properly oriented image, such that skin of the biopsy is at a top of the valid image; input the properly oriented image into a skin and tumor segmentation machine learning model; identify and segment, based on an application of the skin and tumor segmentation machine learning model, the skin and the mass in the properly oriented image to form a marked image; determine a lateral margin value and a deep margin value based on the segmented skin and the mass shown in the marked image; and generate computer-executable instructions configured to cause a user computing device to construct and display a graphical user interface (GUI) that presents the lateral margin value and the deep margin value in association with the marked image.

In some aspects, the techniques described herein relate to a non-transitory computer-readable medium, the non-transitory computer-readable medium storing instructions which, when executed by one or more processors of a computing system, cause the one or more processors to: receive an image including one or more biopsies from a database; input the image into a biopsy segmentation machine learning model; identify and segment, based on an application of the biopsy segmentation machine learning model, a biopsy of the one or more biopsies in the image to form a base image including the biopsy, wherein the biopsy includes a mass and surrounding tissue; input the base image into an ambiguity classifier machine learning model; determine, based on an application of the ambiguity classifier machine learning model, the mass in the base image is distinct from the surrounding tissue; classify, based on the application of the ambiguity classifier machine learning model, the base image as a valid image; input the valid image into a rotation classifier machine learning model; determine, based on an application of the rotation classifier machine learning model, a rotation angle associated with the valid image; rotate the valid image based on the rotation angle to form a properly oriented image, such that skin of the biopsy is at a top of the valid image; input the properly oriented image into a skin and tumor segmentation machine learning model; identify and segment, based on an application of the skin and tumor segmentation machine learning model, the skin and the mass in the properly oriented image to form a marked image; determine a lateral margin value and a deep margin value based on the segmented skin and the mass shown in the marked image; and generate computer-executable instructions configured to cause a user computing device to construct and display a graphical user interface (GUI) that presents the lateral margin value and the deep margin value in association with the marked image.

### BRIEF DESCRIPTION OF THE DRAWINGS

The accompanying drawings, which are incorporated in and constitute a part of this specification, illustrate various example embodiments and together with the description, serve to explain the principles of the disclosed embodiments.

FIG. 1 is an exemplary diagram of a system for automating tumor margin assessment, according to aspects of the disclosure.

FIG. 2 is an exemplary diagram of the ML models used for automating tumor margin assessment, according to aspects of the disclosure.

FIGS. 3A and 3B are a flowchart of a process for automating tumor margin assessment, according to aspects of the disclosure.

FIG. 4 illustrates an exemplary process for automating tumor margin assessment, according to aspects of the disclosure.

FIG. 5 illustrates rotating an image of a biopsy, according to aspects of the disclosure.

FIG. 6A illustrates a process for calculating the lateral margin measurement of a biopsy, according to aspects of the disclosure.

FIG. 6B illustrates a process for calculating the deep margin measurement of a biopsy, according to aspects of the disclosure.

FIG. 7 shows an image of a biopsy with lateral and deep margin measurements, according to aspects of the disclosure.

FIG. 8 shows an exemplary machine learning training flow chart.

FIG. 9 illustrates an implementation of a computer system that executes techniques presented herein.

### DETAILED DESCRIPTION OF EMBODIMENTS

Various non-limiting embodiments of the present disclosure will now be described to provide an overall understanding of the principles of the structure, function, and use of systems and methods disclosed herein for employing machine learning (ML) methodologies for automating the assessment of tumor margin measurements.

The analysis of biopsies presents a multitude of challenges, as it can be a time-consuming and non-standardized process. Surgical pathologists are tasked with performing the analysis and creating a report based on the findings. Specifically, for biopsies of cutaneous masses, part of the analysis includes measuring the lateral and deep margins, which is often labor-intensive. The lateral and deep margins define the distance between the edge of the mass (e.g., tumor) and the edge of the biopsy. It is critical to determine the amount of healthy tissue between the mass and surgical incision as it can help predict mass reoccurrence. Surgical margin assessment, while performed solely by pathologists, is technically straightforward; pathologists would benefit from automation of this time-consuming work.

The present disclosure discusses techniques for addressing the aforementioned challenges, for example by leveraging advanced methodologies from fields such as digital pathology, medical technology, and ML. The integration of ML throughout the process of measuring lateral and deep margins, as described in the present disclosure, significantly expedites the process, such as identifying various tissue types, properly orienting the biopsy, determining if the biopsy is clear enough to analyze, and defining the lateral and deep margins. The techniques disclosed herein offer unprecedented capabilities for rapid and accurate analysis of the biopsies, facilitating the determination of lateral and deep margins. Further, the disclosed ML-driven algorithms streamline biopsy analysis and provide unique insights, such as the chance of reoccurrence. Moreover, the ML-driven algorithms lessen the workload for pathologists and allow pathologists to focus on creating reports instead of making measurements.

The present disclosure provides embodiments that lead to significant technical advancements in the field of digital pathology. System 100 discussed in the present disclosure overcomes technical shortcomings of conventional techniques by, for example, preparing and analyzing a digital medical image of a biopsy to determine lateral and deep margins and subsequently outputting the analyzed biopsy so pathologists can review the analyzed biopsy. This entails harnessing large datasets encompassing histology slides with various masses from various anatomic sites to train models capable of determining lateral and deep margins. Additionally, integrating real-time data streams and feedback mechanisms into the biopsy analysis process enables continuous refinement of predictive models, ensuring they remain robust and adaptive in the face of evolving trends and procedures in the field. Through this technical advancement, pathologists can streamline their review of biopsies and reduce time spent on making lateral and deep margin measurements, allowing for an increase in useful data to determine, for example, the chance of reoccurrence of the tumor.

The above technical improvements, and additional technical improvements, will be described in detail throughout the present disclosure. Also, it should be apparent to a person of ordinary skill in the art that the technical improvements of the embodiments provided by the present disclosure are not limited to those explicitly discussed herein, and that additional technical improvements exist.

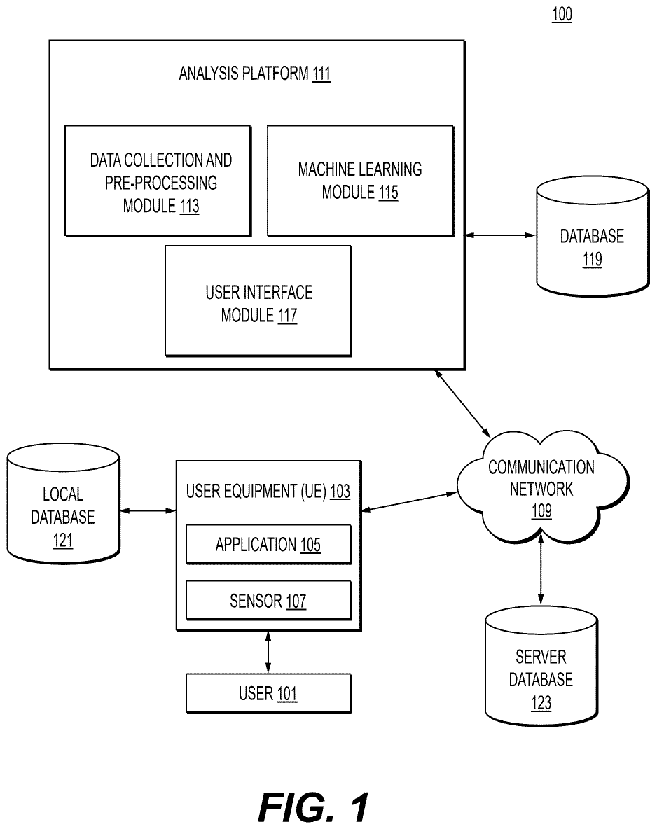

The present disclosure contemplates implementing ML methodologies, along with other techniques, for accelerating the tumor margin measurement process. Specifically, FIG. 1 is a diagram showing an example of a system using various techniques to automate tumor margin assessment. FIG. 1 includes the system 100 that comprises a user equipment (UE) 103 (interacted with by a user 101) that includes application(s) 105 and sensor(s) 107, a communication network 109, an analysis platform 111, and databases 119, 121, and 123.

In one instance, the user 101 is a pathologist that engages with the system 100. Generally, user 101 is a medical professional capable and authorized to analyze biopsies. In aspects of the present disclosure, user 101 accesses the analysis platform 111 via application 105 on UE 103. That is, analysis platform 111 may be downloaded on UE 103. In other aspects, user 101 accesses the analysis platform 111 via communication network 109. For example, analysis platform 111 is accessed from a cloud server, a local server, etc. In one instance, the histology slides are collected through various data collection mechanisms that collect data from a plurality of data sources (e.g., the database 119, the local database 121, the server database 123, the UE 103, and/or any other databases necessary).

In one instance, the UE 103 includes, but is not restricted to, any type of mobile terminal, wireless terminal, fixed terminal, or portable terminal. Examples of the UE 103, include, but are not restricted to, a mobile handset, a wireless communication device, a station, a unit, a device, a multimedia computer, a multimedia tablet, an Internet node, a communicator, a desktop computer, a laptop computer, a notebook computer, a netbook computer, a tablet computer, a Personal Communication System (PCS) device, a personal navigation device, a Personal Digital Assistant (PDA), a digital camera/camcorder, an infotainment system, a dashboard computer, a television device, or any combination thereof, including the accessories and peripherals of these devices, or any combination thereof. In addition, the UE 103 facilitates various input means for receiving and generating information, including, but not restricted to, a touch screen capability, a keyboard, and keypad data entry, a voice-based input mechanism, and the like. Any known and future implementations of the UE 103 are also applicable. As an example, UE 103 may be a work computer used by a pathologist that is configured to run software for the analysis platform 111.

In one instance, the application 105 includes various applications such as, but not restricted to, content provisioning applications, software applications, networking applications, multimedia applications, media player applications, camera/imaging applications, storage services, contextual information determination services, location-based services, notification services, social networking services, and the like. In one embodiment, one of the applications 105 at the UE 103 acts as a client for the analysis platform 111 and performs one or more functions associated with the functions of the analysis platform 111 by interacting with the analysis platform 111 over the communication network 109. In one example, UE 103 receives data and stores the data in a local database 121 (e.g., a database of histology slides) such that, over time, local database 121 compiles data from UE 103 to provide to analysis platform 111. In the same example or a different example, UE 103 uploads data to server database 123 (e.g., a database of histology slides) via communication network 109. Server database 123 stores data from one or more UE 103 to generate a data set for analysis platform 111. In this way, data for system 100 is stored in local database 121 and/or server database 123. The database 119 (e.g., a database of histology slides) then used by the analysis platform 111 includes local database 121, server database 123 and/or other databases necessary such as a system database, historical database, etc.

By way of example, the sensor 107 includes any type of sensor. In one instance, the sensors 107 include, for example, a network detection sensor for detecting wireless signals or receivers for different short-range communications (e.g., Bluetooth, Wi-Fi, Li-Fi, near field communication (NFC), etc.), a global positioning sensor for gathering location data, a camera/imaging sensor for gathering image data, an audio recorder for gathering audio data, and the like. In another instance, the sensors 107 include, for example, inertial measurement unit (IMU) sensors, electrocardiogram (ECG) sensors, sensors to detect blood glucose level, sensors to measure respiration rate, heart rate detection sensors (e.g., optical Heart Rate (PPG) sensor), sensors to monitor body temperature, micro-electro-mechanical system (MEMS) based miniature motion sensors, gyroscope, accelerometer, magnetometer, infrared sensor, microphone, gas sensor, etc. In one example sensors 107 include any type of sensor necessary to facilitate receiving information for analysis and/or providing information to analysis platform 111 via communication network 109.

In one instance, various elements of the system 100 communicate with each other through the communication network 109. The communication network 109 supports a variety of different communication protocols and communication techniques. In one embodiment, the communication network 109 allows the analysis platform 111 to communicate with the UE 103. The communication network 109 of the system 100 includes one or more networks such as a data network, a wireless network, a telephony network, or any combination thereof. It is contemplated that the data network is any local area network (LAN), metropolitan area network (MAN), wide area network (WAN), a public data network (e.g., the Internet), short range wireless network, or any other suitable packet-switched network, such as a commercially owned, proprietary packet-switched network, e.g., a proprietary cable or fiber-optic network, and the like, or any combination thereof. In addition, the wireless network is, for example, a cellular communication network and employs various technologies including 5G (5th Generation), 4G, 3G, 2G, Long Term Evolution (LTE), wireless fidelity (Wi-Fi), Bluetooth®, Internet Protocol (IP) data casting, satellite, mobile ad-hoc network (MANET), vehicle controller area network (CAN bus), and the like, or any combination thereof.

In one embodiment, the analysis platform 111 is a platform with multiple interconnected components. The analysis platform 111 includes one or more servers, intelligent networking devices, computing devices, components, and corresponding software for formatting digital medical images (e.g., histology slides) and measuring lateral and deep margins. In addition, it is noted that the analysis platform 111 may be a separate entity of the system 100, or, as previously mentioned, analysis platform 111 may be on UE 103.

The analysis platform 111 identifies and segments the biopsies present on each digital medial image (e.g., histology slide) provided. Each biopsy is casted to its own image and the analysis platform 111 determines if the tissue is clear enough to measure. The biopsy may be a biopsy from a pet, such as a dog or a cat, or the biopsy may be a biopsy from a human. In general, the biopsy may be from any mammal. The analysis platform 111 rotates the biopsy until the skin surface is at the top of the image and the body is at the bottom of the image. Once correctly formatted, the analysis platform 111 identifies and segments any skin and tumor tissue that is present to then measure the lateral and deep margins. The analysis platform 111 may further analyze the lateral and deep margins to determine a chance of reoccurrence of the tumor.

In one embodiment, the analysis platform 111 includes a data collection and pre-processing module 113, a machine learning module 115, a user interface module 117, or any combination thereof. As used herein, terms such as “component” or “module” generally encompass hardware and/or software, e.g., that a processor or the like used to implement associated functionality. It is contemplated that the functions of these components are combined in one or more components or performed by other components of equivalent functionality.

In one embodiment, the data collection and pre-processing module 113 collects relevant data, as described above, for analysis by analysis platform 111. In one embodiment, the data collection and pre-processing module 113 uses a web-crawling component to access various databases (e.g., database 119, local database 121, server database 123), or other information sources (e.g., third-party databases), to collect relevant data. In one embodiment, the data collection and pre-processing module 113 includes various software applications (e.g., data mining applications in Extended Meta Language (XML)) that automatically search for and return relevant data. In one example, the data collection and pre-processing module 113 collects data provided by user 101. The data provided by user 101 includes digital medical images (e.g., histology slides) containing masses with associated skin margins. The images contain various types of masses from a variety of anatomic sites. As will be further described below, data collection and pre-processing module 113 may include a plurality of machine learning models, each of which may require a separate data set to train. The data sets may be subsets of relevant histology slides from the data provided by user 101 or from one of databases 119, 121, 123. In one embodiment, the collection of relevant data is automated.

In some aspects, the data collection and pre-processing module 113 purges unusable or unreliable images from the provided data. For example, the data collection and pre-processing module 113 may be configured to remove incomplete histology slides or corrupted files from the data. The data collection and pre-processing module 113 may standardize the data provided. For example, a thumbnail of each histology slide may be extracted and subsequently converted to a PNG file, JPEG file, or the like. Duplicate histology slides may also be identified and removed by the data collection and pre-processing module 113. In general, data collection and pre-processing module 113 may perform all functions necessary to provide properly formatted data (e.g., histology slides) to machine learning module 115.

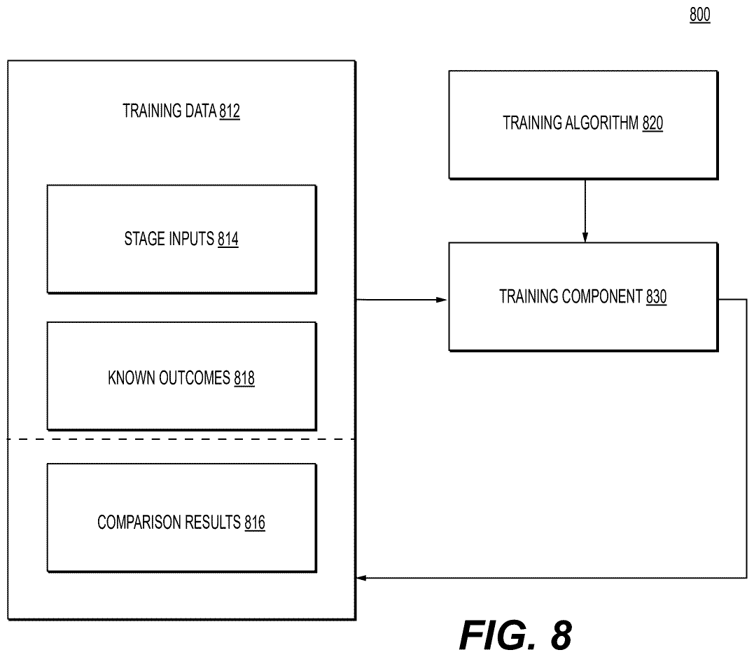

In one embodiment, the machine learning module 115 is configured for unsupervised machine learning that does not require training using known outcomes 818, as described below and shown in FIG. 8. Unsupervised machine learning utilizes machine learning algorithms to analyze and cluster unlabeled data sets and discover hidden patterns or data groupings (e.g., similarities and differences within data), without supervision. In one example, unsupervised machine learning techniques implement approaches that include clustering (e.g., deep embedded clustering, K-means clustering, hierarchical clustering, probabilistic clustering), association rules, classification, principal component analysis (PCA), or the like. The machine learning module 115 utilizes unsupervised machine learning techniques to identify target data entries and predict target data entries with anomalies.

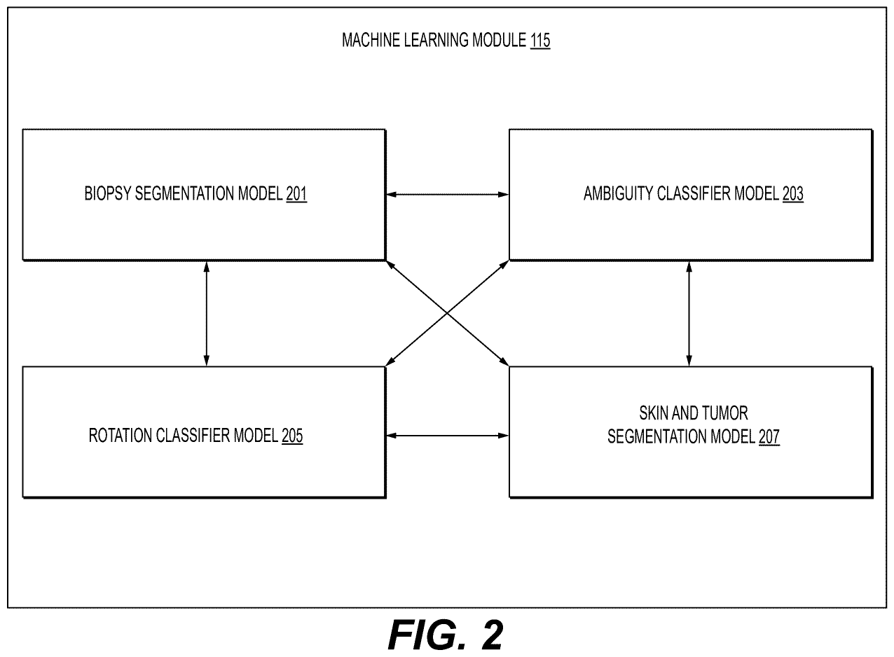

In one embodiment, the machine learning module 115 is additionally or alternatively configured for supervised machine learning techniques that utilize training data (e.g., training data 812 illustrated in the training flowchart 800 of FIG. 8), for training a machine learning model or models configured to analyze biopsies in digital medical images and determine lateral and deep margin measurements. In one example, the machine learning module 115 performs model training using training data, e.g., data from other modules that contains input and correct output, to allow the model to learn over time. The training is performed based on the deviation of a processed result from a documented result when the inputs are fed into the machine learning model, e.g., an algorithm measures accuracy through a loss function, adjusting until the error has been sufficiently minimized. In one embodiment, the machine learning module 115 randomizes the ordering of the training data, visualizes the training data to identify relevant relationships between different variables, identifies any data imbalances, and splits the training data into two parts where one part is for training a model and the other part is for validating the trained model, de-duplicating, normalizing, correcting errors in the training data, and so on. The machine learning module 115 implements various machine learning techniques, e.g., K-nearest neighbors, cox proportional hazards model, decision tree learning, association rule learning, neural network (e.g., recurrent neural networks, graph convolutional neural networks, deep neural networks), regression, inductive programming logic, support vector machines, Bayesian models, Gradient boosted machines (GBM), LightGBM (LGBM), Xtra tree classifier, etc. The numerous machine learning models of machine learning module 115 are further described with respect to FIG. 2.

In one embodiment machine learning module 115 transmits the analyzed biopsy images and results to the user interface module 117. The user interface module 117 enables a presentation of a graphical user interface (GUI) in the UE 103 that facilitates notifications and visualizations of the biopsies in the histology slides and enables a presentation of a GUI for the user 101. The user interface module 117 employs various application programming interfaces (APIs) or other function calls corresponding to the application 105 on the UE 103, thus enabling the display of graphics primitives such as icons (e.g., flags), measurements, boundary boxes, bar graphs, menus, buttons, data entry fields, groups of data entries, lists, etc. In another embodiment, the user interface module 117 causes interfacing of guidance information to include, at least in part, one or more annotations, audio messages, video messages, or a combination thereof pertaining to the notification (e.g., a notification of lateral and deep margins). In one example embodiment, the user interface module 117 operates in connection with augmented reality (AR) processing techniques, wherein various applications, graphic elements, and features interact to present anomaly notifications in a format that is understandable by the recipients (e.g., user 101 such as a pathologist). The results presented by user interface module 117 may be viewed in ImageScope or another slide viewing application.

The above-described modules and components of the analysis platform 111 are implemented in hardware, firmware, software, or a combination thereof. Though depicted as a separate entity in FIG. 1, it is contemplated that the analysis platform 111 is also implemented for direct operation by the respective UE 103. As such, the analysis platform 111 generates direct signal inputs by way of the operating system of the UE 103. In another embodiment, one or more of the modules 113, 115, 117 are implemented for operation by the respective UEs, as the analysis platform 111. The various executions presented herein contemplate any and all arrangements and models.

In one embodiment, databases 119, 121, 123 include a machine-learning based training database with a pre-defined mapping defining a relationship between various input parameters and output parameters based on various statistical methods. For example, the training database includes machine-learning algorithms to learn mappings between input parameters related to the histology slides, tracing masses and surrounding tissue in biopsies, and lateral and deep margins. The training database is routinely updated and/or supplemented based on machine learning methods.

FIG. 2 is an exemplary diagram of the ML modules used for automating tumor margin assessment, according to aspects of the disclosure. Machine learning module 115 may include a biopsy segmentation model 201, an ambiguity classifier model 203, a rotation classifier model 205, and a skin and tumor segmentation model 207. Models 201, 203, 205, 207 may be provided in any order.

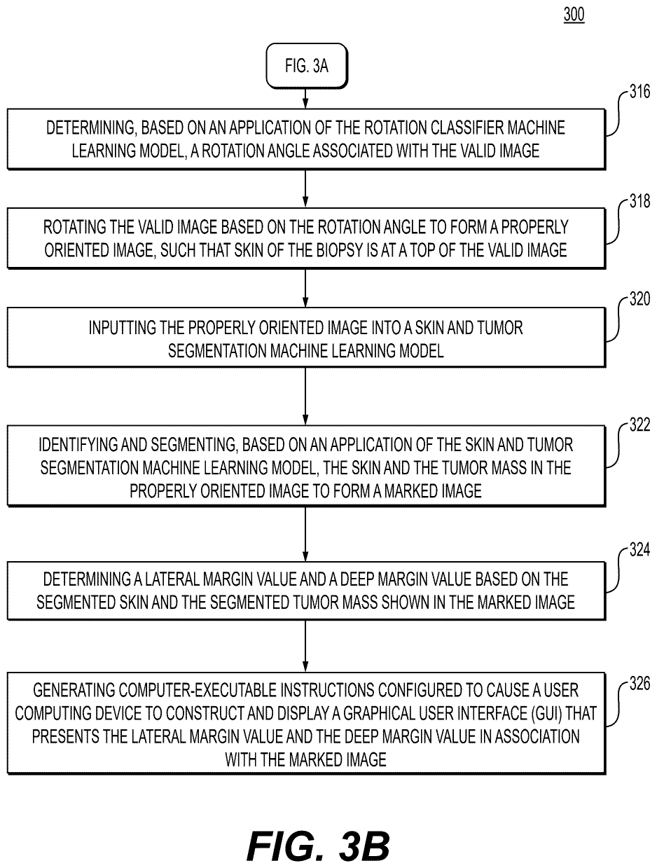

Biopsy segmentation model 201 may be a deep learning model such as a transformer-based segmentation model or multi-task segmentation model. For example, biopsy segmentation model 201 may be based on the Mask2Former architecture. The biopsy segmentation model 201 runs inference to identify and segment the biopsies present on each histology slide. The biopsy segmentation model 201 may learn to annotate the biopsies (e.g., the red boxes around the biopsies seen in slide 422 of FIG. 4) by using a segmentation model such as, for example, Facebook's Segment Anything model. This model may be used to bootstrap a model due to the difficulties of accurately tracing thousands of masses and surrounding normal tissue manually. The annotations generated by biopsy segmentation model 201 include coordinates such that biopsy segmentation model 201 can cast each biopsy to its own base image. The base image may be an image of a single biopsy with only the tissue (e.g., both the mass and surrounding normal tissue) visible. Thus, models 203, 205, 207 use the base image to measure the lateral and deep margins for each biopsy.

Ambiguity classifier model 203 may be a deep learning model such as a residual neural network, a residual-based deep learning model, or a deep convolutional neural network with residual connections. For example, ambiguity classifier model 203 may be built on a Resnet-18 architecture. In general, ambiguity classifier model 203 is a binary classifier used to predict whether a biopsy is clear or ambiguous. A biopsy may be considered clear if there is ample contrast between skin, tumor, fat, muscle tissue, etc. in the biopsy. Conversely, a biopsy may be considered ambiguous if the mass is not distinct enough from the surrounding normal tissue. The margin measurements cannot be determined for ambiguous biopsies since it is not possible to apply measurement rules to find the margins of the mass if the mass blends in with the surrounding normal tissue. FIG. 4 shows several examples of clear biopsies 442 and ambiguous biopsies 444 that illustrate the contrast (in clear biopsies 442) between the mass and the tissue or illustrate the lack of contrast (in ambiguous biopsies 444) between the mass and the tissue. Ambiguity classifier model 203 may be trained to classify the biopsies as clear or ambiguous by providing training biopsies that have been manually, automatically, or with minimal supervision labeled as clear or ambiguous. Ambiguous biopsies may be discarded or appropriately marked so a pathologist can separately review the ambiguous biopsies.

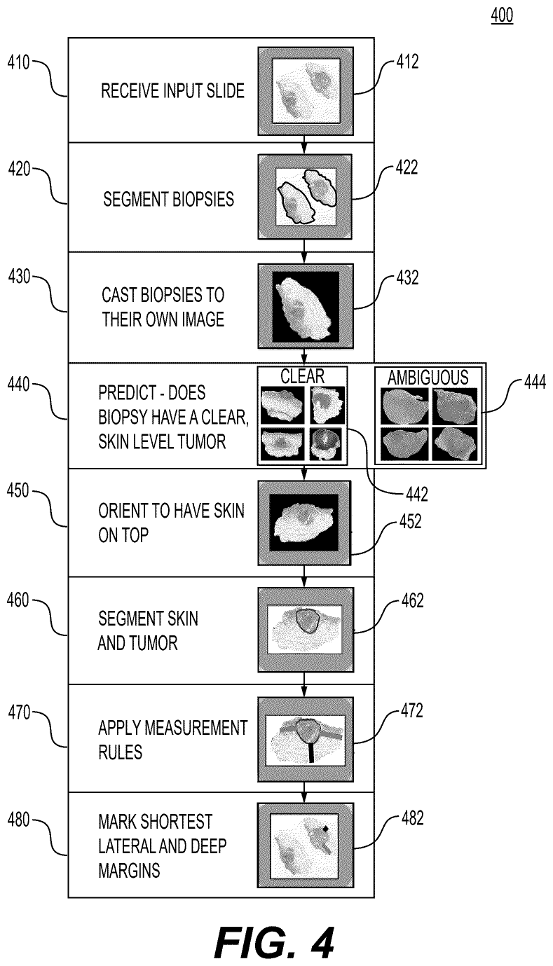

The rotation classifier model 205 may be a deep learning model such as an efficient convolutional neural network or scalable convolutional neural network. For example, rotation classifier model 205 may use an EfficientNet-b5 architecture. The rotation classifier model 205 detects how much to rotate the base image such that the skin surface of the biopsy is at the top of the slide and the body is at the bottom of the slide. In some embodiments, the data set for rotation classifier model 205 may be built by labeling images that are gathered and rotated to be aligned. This is further described with respect to FIG. 5.

Similar to biopsy segmentation model 201, skin and tumor segmentation model 207 may be a deep learning model based on, for example, Mask2Former architecture. The skin and tumor segmentation model 207 may identify and segment any skin and tumor tissue that is present. In some aspects, skin and tumor segmentation model 207 may facilitate outlining the skin (or the periphery of the skin) as one color (e.g., green) and the tumor (or the periphery of the tumor) as another color (e.g., blue). In other aspects, skin and tumor segmentation model 207 may identify the skin and tumor with labels. It is contemplated in the present disclosure that skin and tumor segmentation model 207 may identify and segment the skin and tumor tissue by any understandable means known in the art (e.g., different colors, hatching, highlighting, labels, etc.).

Once the biopsy has been inputted into each of models 201, 203, 205, 207 and the output has been properly prepared, analysis platform 111 determines the lateral and deep margins. For example, separate rule-based methods may be used to measure the lateral and deep margins. In some examples, machine learning module 115 may measure the margins. Any one of or any combination of the ML models described above may determine the margins, annotate the biopsy with the margins, and/or analyze the margins to determine the risk of tumor reoccurrence.

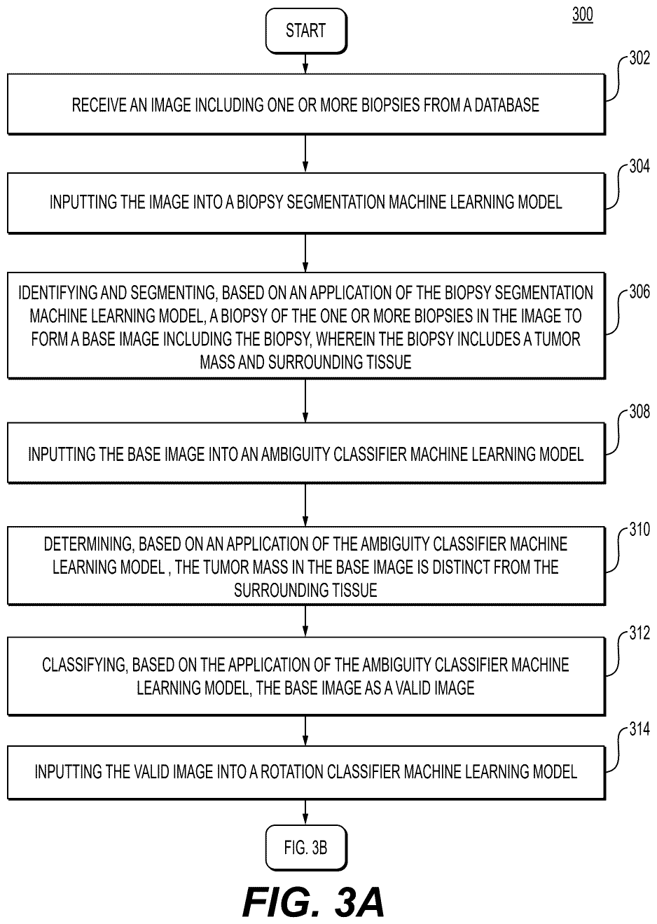

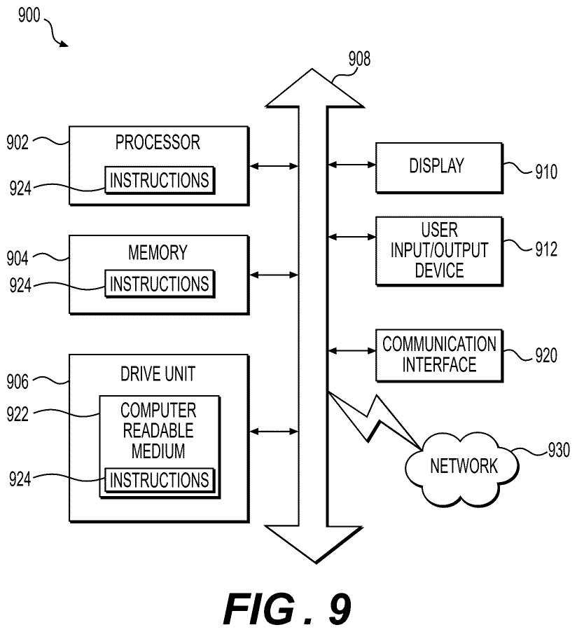

FIGS. 3A and 3B are a flowchart of a process for automating tumor margin assessment using one or more machine learning models, according to aspects of the disclosure. In various embodiments, the analysis platform 111 and/or any of the modules 113, 115, 117 performs one or more portions of the process 300 and are implemented using, for instance, a chip set including a processor and a memory as shown in FIG. 9. As such, the analysis platform 111 and/or any of modules 113, 115, 117 provide means for accomplishing various parts of the process 300, as well as means for accomplishing embodiments of other processes described herein in conjunction with other components of the system 100. Although the process 300 is illustrated and described as a sequence of steps, it is contemplated that various embodiments of the process 300 are performed in any order or combination and need not include all of the illustrated steps.

In step 302, the data collection and pre-processing module 113 of analysis platform receives an image including one or more biopsies from a database (e.g., databases 119, 121, 123). The images may include skin, tumor, fat, and muscle tissue. The pre-processing module 113 receives the slides of the biopsies in an SVS format and converts them into thumbnails.

In step 304, the converted images are inputted into machine learning module 115. Specifically, the images are inputted into biopsy segmentation model 201. In step 306, the biopsy segmentation model 201 identifies and segments each biopsy present in the inputted image. The segmented biopsy forms a base image, including both mass and surrounding tissue.

In step 308, the base image is inputted into ambiguity classifier model 203. In step 310, the ambiguity classifier model 203 determines the mass in the base image is distinct from the surround tissue. For example, ambiguity classifier model 203 may classify the base image as a clear image or an ambiguous image depending on the level of contrast present. In step 312, the ambiguity classifier model 203 classifies the base image as a valid image.

In step 314, the valid image is inputted into rotation classifier model 205. In step 316, the rotation classifier model 205 determines a rotation angle associated with the valid image. The rotation classifier model 205 may determine the rotation angle as a multiple of a specific value. For example, the rotation classifier model 205 may determine the valid image needs to be rotated 15 degrees, 30 degrees, 45 degrees, and so forth. Once the correct value is determined, the valid image may then be rotated. Alternatively, it is contemplated that the valid image is rotated every interval (e.g., at 15 degrees, 30 degrees, etc.) and the rotation classifier model 205 is configured to check if the valid image is in the optimal orientation after each rotation. Specifically, in step 318, the rotation classifier model 205 is configured to rotate the valid image based on the rotation angle to form a properly oriented image. An image of a biopsy may be considered properly oriented when the skin of the biopsy is at the top of the image. In some aspects, a properly oriented image is an image where the skin is closest to the top of the image given the rotation angle increment.

In step 320, the properly oriented image is inputted into skin and tumor segmentation model 207. In step 322, the skin and tumor segmentation model 207 identifies and segments the skin and the mass in the properly oriented image to form a marked image. Skin and tumor segmentation model 207 may provide boundary boxes around both the skin and the mass. Based on the output from the models 201, 203, 205, 207, in step 324, a lateral margin value and a deep margin value may be determined based on the segmented skin and the segmented mass shown in the marked image.

In step 326, computer-executable instructions are generated via user interface module 117. The user interface module 117 is configured to cause a user computing device (e.g., UE 103 or a separate device connected via communication network 109) to construct and display a graphical user interface (GUI) that presents the lateral margin value and the deep margin value in association with the marked image. As described above, any GUI known in the field is contemplated in the present disclosure. In an example, analysis platform 111 is software downloaded on UE 103 such that the GUI is presented on a screen of UE 103 when accessing the software.

FIG. 4 illustrates an exemplary process for automating tumor margin assessment, according to aspects of the disclosure. Process 400 may begin with the system 100 receiving the input slide 412 at step 410. System 100 may receive input slides 412 in an SVS format. A thumbnail level view of each of the input slides 412 is saved as a PNG, JPEG, or other raster image file types. The saved image file is then provided to machine learning module 115, specifically biopsy segmentation model 201.

The biopsies in slide 422 are identified and segmented at step 420 via biopsy segmentation model 201. The segmented biopsies are split into separate slides so the analysis platform 111 can analyze each individual biopsy. As seen at step 430, each of the individual biopsy slides is casted to a base image 432, which may also be done by biopsy segmentation model 201. The base image 432 may include a plain background (e.g., a solid white or solid black color) with the biopsy overlaid on top. Thus, base image 432 is an image of only the biopsy (e.g., the mass and surrounding tissue).

Once casted to a base image, the biopsy is passed through ambiguity classifier model 203 to determine if the tissue is clear enough to measure. Valid or clear biopsies 442 may have ample contrast between the different parts of the base image (e.g., mass, skin, fat, muscle tissue, background, etc.) so the margins of the mass and surrounding tissue can be accurately determined. Base image 432 may be classified as a clear biopsy 442 or an ambiguous biopsy 444 depending on the clarity of the biopsy. Alternatively, the classification of the biopsy into a clear or an ambiguous category may be performed at an initial stage such as, for example, even before segmenting the biopsies at step 420. However, it should be apparent to a person of ordinary skill in the art that this classification step may be implemented at any suitable stage during the automated tumor margin assessment process.

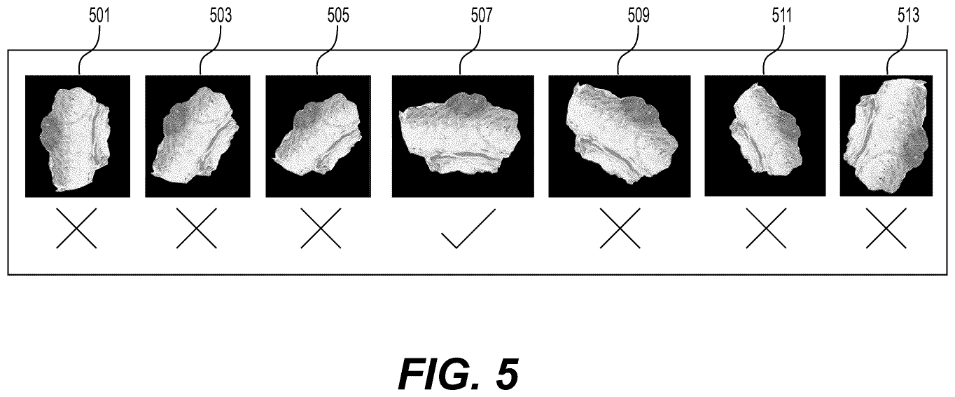

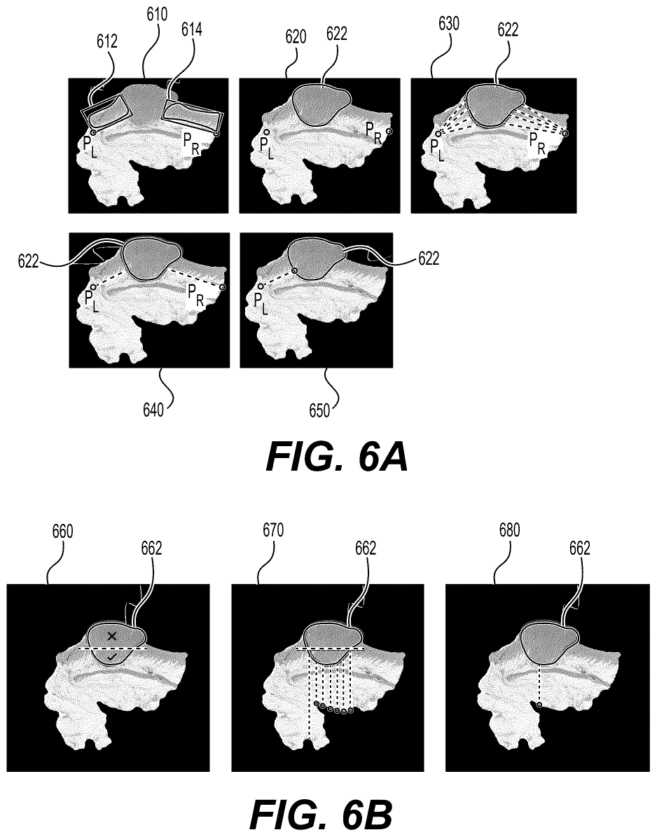

Clear biopsies 442 may then be rotated at step 450 to be a properly oriented image 452 (e.g., skin surface at the top of the slide); this is further described below. Step 460 may identify and segment the skin and tumor present in properly oriented image 452 via skin and tumor segmentation model 207. Specifically, skin and tumor segmentation model 207 may be able to identify two polygon objects and list the coordinates along the perimeter of the polygon to segment the skin and the tumor. Doing so, as seen in segmented biopsy 462, allows the analysis platform 111 to measure the margins as the boundaries of the skin and the mass have been calculated and marked. The measurement rules for the lateral and deep margins may be applied to segmented biopsy 462 at step 470 to determine the margins as seen in slide 472. This is further described with respect to FIGS. 6A and 6B.

After calculating the lateral and deep margins, the margins are marked on the slide. Slide 482 of step 480 shows the biopsy with the determined lateral and deep margins marked for a pathologist to receive. To share slide 482 with the user 101, an XML file may be generated and uploaded to the file system of user 101. For example, the analysis platform 111 may send an XML (or similar) file to UE 103 via communication network 109. Alternatively, analysis platform 111 may be located on UE 103 and, thus, the file is sent and saved locally. The file displays the measurement details in a clinical viewing application for the user 101 (e.g., a pathologist).

The user 101 may review the measurements and determine if the predictions from analysis platform 111 are accurate. If user 101 agrees with the outputted file provided by analysis platform 111, the user 101 may use the lateral and deep margin values to create a report. Process 400 may further include predicting a level of risk related to the chance of the tumor reoccurring. For example, a biopsy with a clean cut around the tumor will have substantially different margin values (e.g., larger values) than a biopsy with a dirty cut around the tumor. Low margin values indicative of a dirty cut may increase the chance of the tumor reoccurring. Analysis platform 111 may be configured to determine this risk. Steps 420-480 may be repeated for each biopsy present on the input slide 412. It is contemplated in the present disclosure that the steps of process 400 may be performed in different orders. For example, step 440 may be performed before step 420 or step 450 may be performed before step 440.

FIG. 5 illustrates rotating an image of a biopsy, according to aspects of the disclosure. The biopsy is considered properly oriented if the skin surface is at the top of the slide while the body is at the bottom of the slide. Initial image 501 is not properly oriented as the skin surface is at the left side of the slide. Rotation classifier model 205 may rotate initial image 501 clockwise in 15-degree increments. Therefore, rotation classifier model 205 may predict one of 24 possible values for the rotation of the biopsy. Rotation classifier model 205 may determine the optimal rotation value prior to the imaging rotating such that the image only rotates once. For example, if the image needs to be rotated 45 degrees, the image may be rotated 45 degrees once instead of 15 degrees three times. Alternatively, each time the image of the biopsy is rotated, rotation classifier model 205 (or analysis platform 111 in general) checks to see if the image is properly oriented. In FIG. 5, each of images 503, 505, 507, 509, 511, 513 is rotated with respect to initial image 501.

Rotation classifier model 205 may have a coarser or finer increment than 15 degrees. In some aspects, rotation classifier model 205 may check if the biopsy is properly oriented after rotating the image of the biopsy 1 degree. In other aspects, rotation classifier model 205 may check after rotating the image of the biopsy 30 degrees. Due to rotation classifier model 205 rotating the biopsy in intervals, the analysis platform 111 is capable of determining which interval position is the closest to properly aligned if none of the interval positions yields a perfect result. For example, if the biopsy is 5 degrees counterclockwise from properly aligned (e.g., the skin surface being at the top of the image and the body at the bottom) in a first interval position, and the biopsy is 10 degrees clockwise from properly aligned in a second interval position, the rotation classifier model 205 will choose the first interval position.

FIG. 6A illustrates a process for calculating the lateral margin measurement of a biopsy, according to aspects of the disclosure. The lateral margin value is determined using the location of skin and tumor tissue while taking into account the orientation of the slide. The analysis platform 111 determines the proper scale of the biopsy to accurately label the lateral and deep margin values with appropriate units (e.g., mm, in, etc.). Initially, the minimum area-bounding box around the skin is calculated with four vertices. In some aspects, the bounding box may only have three vertices. In other aspects, the bounding box may have more than four vertices. This is seen with left box 612 and right box 614 in slide 610. Left box 612 may surround a first portion of the skin while right box 614 may surround a second portion of the skin. The analysis platform 111 considers each of the four vertices of left box 612 and right box 614 and selects the vertex (e.g., point) that is underneath the skin (e.g., within the boundaries of the biopsy and below the skin) and furthest away from the mass. For example, as seen in slide 610, P_{L} is selected for left box 612 and P_{R} is selected for right box 614. P_{L} and P_{R} are considered anchor points and are used to determine the lateral margin measurement.

Slide 620 illustrates the anchor points P_{L} and P_{R} on the biopsy with the tumor 622 segmented by creating a boundary box around the perimeter. For each lateral point, the shortest straight-line distance between tumor 622 and P_{L} or P_{R} is calculated. Analysis platform 111 finds the nearest point on the boundary of tumor 622 to P_{L} and the nearest point on the boundary of tumor 622 to P_{R}. Slide 630 shows a plurality of lines originating from the anchor points P_{L} and P_{R} that terminate once they touch the boundary of tumor 622.

The shortest line of the plurality of lines is determined and selected for both the right side lateral margin and the left side lateral margin as seen in slide 640. In this way, analysis platform 111 calculates the shortest distance tumor 622 is from the leftmost part of the skin and the shortest distance tumor 622 is from the rightmost part of the skin. The distance between tumor 622 and P_{L} is then compared to the distance between tumor 622 and P_{R} to determine which of the two values is smaller (e.g., the shorter distance). Since only one lateral measurement is returned per biopsy, analysis platform 111 uses the shorter distance of the two calculated. In the example illustrated in FIG. 6A, the distance from anchor point P_{L} to tumor 622 is shorter than the distance from anchor point P_{R} to tumor 622, so only the line from P_{L} to tumor 622 is used as the lateral margin value and shown in slide 650. In some examples, only one of P_{L} or P_{R} may be calculated.

FIG. 6B illustrates a process for calculating the deep margin measurement of a biopsy, according to aspects of the disclosure. The deep margin value is calculated using the location of the tumor and the orientation of the biopsy to search downwards into the body. Only the lowest (e.g., the deepest into the body) 30% (or any other suitable percentage) of points belonging to the tumor segmentation (e.g., the segmentation performed by skin and tumor segmentation model 207) are marked as eligible for measurement, which is seen in slide 660. Tumor 662 is split into two sections; one section being ineligible for measurement (e.g., the highest 70% of points belonging to the perimeter of tumor 662 indicated with an “x”) and one section being eligible for measurement (e.g., the lowest 30% of points belonging to the perimeter of tumor 662 indicated with a check mark).

For each eligible perimeter point, a straight-line distance traveling downwards into the body of the biopsy between the perimeter point and the edge of the biopsy is calculated. Slide 670 shows several sample distance lines drawn. As seen, these distance lines are straight down from a point on the perimeter of tumor 662 until the distance line comes to an edge of the biopsy. Since only one deep margin value is stored and provided to user 101, the analysis platform 111 determines which distance line (e.g., which point on the perimeter of tumor 662) provides the shortest distance, as seen in slide 680. The shortest distance form a point of the segmented tumor 662 to an edge of the biopsy is used as the deep margin value and shown to user 101.

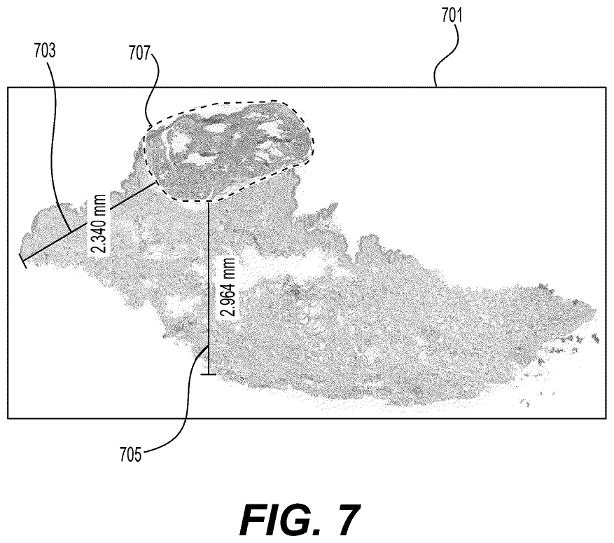

FIG. 7 shows an image of a biopsy with lateral and deep margin measurements, according to aspects of the disclosure. Slide 701 shows a biopsy with a line representing the lateral margin 703 calculated from tumor 707 and a line representing the deep margin 705 calculated from tumor 707. Lateral margin 703 is the margin between the boundary of tumor 707 and the leftmost point of the skin. In this example, lateral margin 703 is measured as 2.340 mm. Deep margin 705 is the margin between the boundary of tumor 707 and the closest edge point of the biopsy below the tumor 707. In this example, deep margin 705 is 2.694 mm. In one or more embodiments, the image of the biopsy provided to user 101 by analysis platform 111 may be substantially similar to FIG. 7. For example, the image of the biopsy provided to user 101 by analysis platform 111 may include a boundary box around the tumor, a line indicating the lateral margin with a corresponding value, and a line indicating the deep margin with a corresponding value.

FIG. 8 shows an exemplary machine learning training flow chart. One or more implementations disclosed herein may include and/or may be implemented using one or more machine learning models. For example, one or more of the modules of the analysis platform 111 are implemented using one or more machine learning models and/or are used to train the one or more machine learning models. A given machine learning model is trained using the training flowchart 800 of FIG. 8. Training data 812 includes one or more of stage inputs 814 and known outcomes 818 related to the machine learning model to be trained. Stage inputs 814 are from any applicable source including text, visual representations, data, values, comparisons, and stage outputs, e.g., one or more outputs from one or more steps from FIGS. 3A and 3B. The known outcomes 818 are included for the machine learning models generated based on supervised or semi-supervised training. An unsupervised machine learning model is not be trained using known outcomes 818. Known outcomes 818 includes known or desired outputs for future inputs similar to or in the same category as stage inputs 814 that do not have corresponding known outputs.

The training data 812 and a training algorithm 820, e.g., one or more of the modules implemented using the machine learning model and/or are used to train the machine learning model, is provided to a training component 830 that applies the training data 812 to the training algorithm 820 to generate the machine learning model. According to an implementation, the training component 830 is provided comparison results 816 that compare a previous output of the corresponding machine learning model to apply the previous result to re-train the machine learning model. The comparison results 816 are used by training component 830 to update the corresponding machine learning model. The training algorithm 820 utilizes machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, classifiers such as K-Nearest Neighbors, and/or discriminative models such as Decision Forests and maximum margin methods, the model specifically discussed herein, or the like.

The one or more machine learning models used herein may be trained and/or used by adjusting one or more weights and/or one or more layers of the one or more machine learning models. For example, during training, a given weight is adjusted (e.g., increased, decreased, removed) based on training data or input data. Similarly, a layer is updated, added, or removed based on training data/and or input data. The resulting outputs are adjusted based on the adjusted weights and/or layers.

FIG. 9 illustrates an implementation of a computer system that executes techniques presented herein. In general, any process or operation discussed in this disclosure is understood to be computer-implementable, such as the processes illustrated in FIGS. 1-7 are performed by one or more processors of a computer system as described herein. A process or process step performed by one or more processors is also referred to as an operation. The one or more processors are configured to perform such processes by having access to instructions (e.g., software or computer-readable code) that, when executed by one or more processors, cause one or more processors to perform the processes. The instructions are stored in a memory of the computer system. A processor is a central processing unit (CPU), a graphics processing unit (GPU), or any suitable type of processing unit.

A computer system, such as a system or device implementing a process or operation in the examples above, includes one or more computing devices. One or more processors of a computer system are included in a single computing device or distributed among a plurality of computing devices. One or more processors of a computer system are connected to a data storage device. A memory of the computer system includes the respective memory of each computing device of the plurality of computing devices.

The computer system 900 includes a set of instructions that are executed to cause the computer system 900 to perform any one or more of the methods or computer based functions disclosed herein. The computer system 900 operates as a standalone device or is connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” refers to any device or portion of a device that processes electronic data, e.g., from registers and/or memory to transform that electronic data into other electronic data that, e.g., is stored in registers and/or memory. A “computer,” a “computing machine,” a “computing platform,” a “computing device,”or a “server”includes one or more processors.

In a networked deployment, the computer system 900 operates in the capacity of a server or as a client user computer in a server-client user network environment, or as a peer computer system in a peer-to-peer (or distributed) network environment. The computer system 900 is also implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 900 is implemented using electronic devices that provide voice, video, or data communication. Further, while the computer system 900 is illustrated as a single system, the term “system” shall also be taken to include any collection of systems or sub-systems that individually or jointly execute a set, or multiple sets, of instructions to perform one or more computer functions.

As illustrated in FIG. 9, the computer system 900 includes a processor 902, e.g., a central processing unit (CPU), a graphics processing unit (GPU), or both. The processor 902 is a component in a variety of systems. For example, the processor 902 is part of a standard personal computer or a workstation. The processor 902 is one or more processors, digital signal processors, application specific integrated circuits, field programmable gate arrays, servers, networks, digital circuits, analog circuits, combinations thereof, or other now known or later developed devices for analyzing and processing data. The processor 902 implements a software program, such as code generated manually (i.e., programmed).

The computer system 900 includes a memory 904 that communicates via bus 908. Memory 904 is a main memory, a static memory, or a dynamic memory. Memory 904 includes, but is not limited to computer-readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 904 includes a cache or random-access memory for the processor 902. In alternative implementations, the memory 904 is separate from the processor 902, such as a cache memory of a processor, the system memory, or other memory. Memory 904 is an external storage device or database for storing data. Examples include a hard drive, compact disc (“CD”), digital video disc (“DVD”), memory card, memory stick, floppy disc, universal serial bus (“USB”) memory device, or any other device operative to store data. The memory 904 is operable to store instructions executable by the processor 902. The functions, acts, or tasks illustrated in the figures or described herein are performed by processor 902 executing the instructions stored in memory 904. The functions, acts, or tasks are independent of the particular type of instruction set, storage media, processor, or processing strategy and are performed by software, hardware, integrated circuits, firmware, micro-code, and the like, operating alone or in combination. Likewise, processing strategies include multiprocessing, multitasking, parallel processing, and the like.

As shown, the computer system 900 further includes a display 910, such as a liquid crystal display (LCD), an organic light emitting diode (OLED), a flat panel display, a solid-state display, a cathode ray tube (CRT), a projector, a printer or other now known or later developed display device for outputting determined information. The display 910 acts as an interface for the user to see the functioning of the processor 902, or specifically as an interface with the software stored in the memory 904 or in the drive unit 906.

Additionally or alternatively, the computer system 900 includes an input/output device 912 configured to allow a user to interact with any of the components of the computer system 900. The input/output device 912 is a number pad, a keyboard, a cursor control device, such as a mouse, a joystick, touch screen display, remote control, or any other device operative to interact with the computer system 900.

The computer system 900 also includes the drive unit 906 implemented as a disk or optical drive. The drive unit 906 includes a computer-readable medium 922 in which one or more sets of instructions 924, e.g. software, is embedded. Further, the sets of instructions 924 embodies one or more of the methods or logic as described herein. Instructions 924 resides completely or partially within memory 904 and/or within processor 902 during execution by the computer system 900. The memory 904 and the processor 902 also include computer-readable media as discussed above.

In some systems, computer-readable medium 922 includes the set of instructions 924 or receives and executes the set of instructions 924 responsive to a propagated signal so that a device connected to network 930 communicates voice, video, audio, images, or any other data over network 930. Further, the sets of instructions 924 are transmitted or received over the network 930 via the communication port or interface 920, and/or using the bus 908. The communication port or interface 920 is a part of the processor 902 or is a separate component. The communication port or interface 920 is created in software or is a physical connection in hardware. The communication port or interface 920 is configured to connect with the network 930, external media, display 910, or any other components in the computer system 900, or combinations thereof. The connection with network 930 is a physical connection, such as a wired Ethernet connection, or is established wirelessly as discussed below. Likewise, the additional connections with other components of the computer system 900 are physical connections or are established wirelessly. Network 930 alternatively be directly connected to the bus 908.

While the computer-readable medium 922 is shown to be a single medium, the term “computer-readable medium” includes a single medium or multiple media, such as a centralized or distributed database, and/or associated caches and servers that store one or more sets of instructions. The term “computer-readable medium” also includes any medium that is capable of storing, encoding, or carrying a set of instructions for execution by a processor or that causes a computer system to perform any one or more of the methods or operations disclosed herein. The computer-readable medium 922 is non-transitory, and may be tangible.

The computer-readable medium 922 includes a solid-state memory such as a memory card or other package that houses one or more non-volatile read-only memories. The computer-readable medium 922 is a random-access memory or other volatile re-writable memory. Additionally or alternatively, the computer-readable medium 922 includes a magneto-optical or optical medium, such as a disk or tapes or other storage device to capture carrier wave signals such as a signal communicated over a transmission medium. A digital file attachment to an e-mail or other self-contained information archive or set of archives is considered a distribution medium that is a tangible storage medium. Accordingly, the disclosure is considered to include any one or more of a computer-readable medium or a distribution medium and other equivalents and successor media, in which data or instructions are stored.

In an alternative implementation, dedicated hardware implementations, such as application specific integrated circuits, programmable logic arrays, and other hardware devices, is constructed to implement one or more of the methods described herein. Applications that include the apparatus and systems of various implementations broadly include a variety of electronic and computer systems. One or more implementations described herein implement functions using two or more specific interconnected hardware modules or devices with related control and data signals that are communicated between and through the modules, or as portions of an application-specific integrated circuit. Accordingly, the present system encompasses software, firmware, and hardware implementations.

Computer system 900 is connected to network 930. Network 930 defines one or more networks including wired or wireless networks. The wireless network is a cellular telephone network, an 902.10, 902.16, 902.20, or WiMAX network. Further, such networks include a public network, such as the Internet, a private network, such as an intranet, or combinations thereof, and utilizes a variety of networking protocols now available or later developed including, but not limited to TCP/IP based networking protocols. Network 930 includes wide area networks (WAN), such as the Internet, local area networks (LAN), campus area networks, metropolitan area networks, a direct connection such as through a Universal Serial Bus (USB) port, or any other networks that allows for data communication. Network 930 is configured to couple one computing device to another computing device to enable communication of data between the devices. Network 930 is generally enabled to employ any form of machine-readable media for communicating information from one device to another. Network 930 includes communication methods by which information travels between computing devices. Network 930 is divided into sub-networks. The sub-networks allow access to all of the other components connected thereto or the sub-networks restrict access between the components. Network 930 is regarded as a public or private network connection and includes, for example, a virtual private network or an encryption or other security mechanism employed over the public Internet, or the like.

In accordance with various implementations of the present disclosure, the methods described herein are implemented by software programs executable by a computer system. Further, in an example, non-limited implementation, implementations can include distributed processing, component/object distributed processing, and parallel processing. Alternatively, virtual computer system processing can be constructed to implement one or more of the methods or functionality as described herein.

Although the present specification describes components and functions that are implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosure is not limited to any particular implementation or programming technique and that the disclosure is implemented using any appropriate techniques for implementing the functionality described herein. The disclosure is not limited to any particular programming language or operating system.

It should be appreciated that in the above description of example embodiments of the present disclosure, various features are sometimes grouped together in a single embodiment, figure, or description thereof for the purpose of streamlining the disclosure and aiding in the understanding of one or more of the various inventive aspects. This method of the present disclosure, however, is not to be interpreted as reflecting an intention that the claimed embodiments require more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of the present disclosure.

Furthermore, while some embodiments described herein include some but not other features included in other embodiments, combinations of features of different embodiments are meant to be within the scope of the present disclosure, and form different embodiments, as would be understood by those skilled in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the present disclosure.

In the description provided herein, numerous specific details are set forth. However, it is understood that embodiments of the present disclosure are practiced without these specific details. In other instances, well-known methods, structures and techniques have not been shown in detail in order not to obscure an understanding of this description.

Thus, while there has been described what are believed to be the preferred embodiments of the present disclosure, those skilled in the art will recognize that other and further modifications are made thereto without departing from the spirit of the present disclosure, and it is intended to claim all such changes and modifications as falling within the scope of the present disclosure. For example, any formulas given above are merely representative of procedures that may be used. Functionality may be added or deleted from the block diagrams and operations may be interchanged among functional blocks. Steps may be added or deleted to methods described within the scope of the present disclosure.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations and implementations are possible within the scope of the disclosure. Accordingly, the disclosure is not to be restricted except in light of the attached claims and their equivalents.

The present disclosure furthermore relates to the following aspects:

Example 1: A computer-implemented method for automating margin measurement for mass removal comprising: receiving, by one or more processors, an image including one or more biopsies from a database; inputting the image, by the one or more processors, into a biopsy segmentation machine learning model; identifying and segmenting, by the one or more processors, based on an application of the biopsy segmentation machine learning model, a biopsy of the one or more biopsies in the image to form a base image including the biopsy, wherein the biopsy includes a mass and surrounding tissue; inputting the base image, by the one more processors, into an ambiguity classifier machine learning model; determining, by the one or more processors, based on an application of the ambiguity classifier machine learning model, the mass in the base image is distinct from the surrounding tissue; classifying, by the one or more processors, based on the application of the ambiguity classifier machine learning model, the base image as a valid image; inputting the valid image, by the one or more processors, into a rotation classifier machine learning model; determining, by the one or more processors, based on an application of the rotation classifier machine learning model, a rotation angle associated with the valid image; rotating, by the one or more processors, the valid image based on the rotation angle to form a properly oriented image, such that skin of the biopsy is at a top of the valid image; inputting the properly oriented image, by the one or more processors, into a skin and tumor segmentation machine learning model; identifying and segmenting, by the one or more processors, based on an application of the skin and tumor segmentation machine learning model, the skin and the mass in the properly oriented image to form a marked image; determining, by the one or more processors, a lateral margin value and a deep margin value based on the segmented skin and the mass shown in the marked image; and generating, by the one or more processors, computer-executable instructions configured to cause a user computing device to construct and display a graphical user interface (GUI) that presents the lateral margin value and the deep margin value in association with the marked image.

Example 2: The method of example 1, further comprising: predicting, by the one or more processors, based on the lateral margin value and the deep margin value, a chance of reoccurrence of the mass.

Example 3: The method of examples 1 or 2, wherein the rotation angle includes zero or more 15-degree increments.

Example 4: The method of any of examples 1-3, wherein the biopsy segmentation machine learning model is a transformer-based segmentation model, and wherein the skin and tumor segmentation machine learning model is a transformer-based segmentation model.

Example 5: The method of any of examples 1-4, wherein the ambiguity classifier machine learning model is a residual neural network, and wherein the rotation classifier machine learning model is a scalable convolutional neural network.

Example 6: The method of any of examples 1-5, wherein determining the lateral margin value comprises: calculating a first minimum area-bounding box around the skin, the first minimum area-bounding box including four vertices; selecting one of the four vertices of the first minimum area-bounding box as a first anchor point, wherein the selected vertex is underneath the skin and is furthest away from the mass out of the four vertices of the first minimum area-bounding box; and calculating a first shortest distance from the first anchor point to the mass, wherein the first shortest distance is the lateral margin value.

Example 7: The method of example 6, wherein the mass separates the skin into a first portion and a second portion, and wherein determining the lateral margin value further comprises: calculating a second minimum area-bounding box around the second portion of the skin, the second minimum area-bounding box including four vertices, wherein the first minimum area-bounding box is around the first portion of the skin; selecting one of the four vertices of the second minimum area-bounding box as a second anchor point, wherein the selected vertex is underneath the skin and is furthest away from the mass out of the four vertices of the second minimum area-bounding box; calculating a second shortest distance from the second anchor point to the mass; and comparing the first shortest distance to the second shortest distance to determine which distance is shorter, and wherein the shorter distance of the first shortest distance and the second shortest distance is the lateral margin value.

Example 8: The method of any of examples 1-7, wherein determining the deep margin value comprises: determining a lowest 30% of points belonging to the segmented mass; and calculating a distance between a point of the lowest 30% of points and an edge of the biopsy directly below the point, wherein the distance is respectively calculated for each point of the lowest 30% of points, and wherein a lowest value of the calculated distances is the deep margin value.

Example 9: The method of any of examples 1-8, wherein the GUI displays the lateral margin value with a lateral margin line, the lateral margin line indicating a lateral margin distance, and wherein the GUI displays the deep margin value with a deep margin line, the deep margin line indicating a deep margin distance.

Example 10: The method of any of example 1-9, wherein the GUI displays a boundary box around the mass, and wherein the GUI displays a chance of reoccurrence of the mass based on the lateral margin value and the deep margin value

Example 11: A system comprising: one or more processors of a computing system; and at least one non-transitory computer-readable medium storing instructions which, when executed by the one or more processors, cause the one or more processors to: receive an image including one or more biopsies from a database; input the image into a biopsy segmentation machine learning model; identify and segment, based on an application of the biopsy segmentation machine learning model, a biopsy of the one or more biopsies in the image to form a base image including the biopsy, wherein the biopsy includes a mass and surrounding tissue; input the base image into an ambiguity classifier machine learning model; determine, based on an application of the ambiguity classifier machine learning model, the mass in the base image is distinct from the surrounding tissue; classify, based on the application of the ambiguity classifier machine learning model, the base image as a valid image; input the valid image into a rotation classifier machine learning model; determine, based on an application of the rotation classifier machine learning model, a rotation angle associated with the valid image; rotate the valid image based on the rotation angle to form a properly oriented image, such that skin of the biopsy is at a top of the valid image; input the properly oriented image into a skin and tumor segmentation machine learning model; identify and segment, based on an application of the skin and tumor segmentation machine learning model, the skin and the mass in the properly oriented image to form a marked image; determine a lateral margin value and a deep margin value based on the segmented skin and the mass shown in the marked image; and generate computer-executable instructions configured to cause a user computing device to construct and display a graphical user interface (GUI) that presents the lateral margin value and the deep margin value in association with the marked image.

Example 12: The system of example 11, the at least one non-transitory computer-readable medium storing instructions which, when executed by the one or more processors, further cause the one or more processors to: predict, based on the lateral margin value and the deep margin value, a chance of reoccurrence of the mass.

Example 13: The system of examples 11 or 12, wherein the rotation angle includes zero or more 15-degree increments.

Example 14: The system of any of examples 11-13, wherein the biopsy segmentation machine learning model is a transformer-based segmentation model, and wherein the skin and tumor segmentation machine learning model is a transformer-based segmentation model.

Example 15: The system of any of examples 11-14, wherein the ambiguity classifier machine learning model is a residual neural network, and wherein the rotation classifier machine learning model is a scalable convolutional neural network.

Example 16: The system of any of examples 11-15, wherein, to determine the lateral margin value, the at least one non-transitory computer-readable medium storing instructions which, when executed by the one or more processors, further cause the one or more processors to: calculate a first minimum area-bounding box around the skin, the first minimum area-bounding box including four vertices; select one of the four vertices of the first minimum area-bounding box as a first anchor point, wherein the selected vertex is underneath the skin and is furthest away from the mass out of the four vertices of the first minimum area-bounding box; and calculate a first shortest distance from the first anchor point to the mass, wherein the first shortest distance is the lateral margin value.

Example 17: The system of example 16, wherein the mass separates the skin into a first portion and a second portion, and wherein, to determine the lateral margin value, the at least one non-transitory computer-readable medium storing instructions which, when executed by the one or more processors, further cause the one or more processors to: calculate a second minimum area-bounding box around the second portion of the skin, the second minimum area-bounding box including four vertices, wherein the first minimum area-bounding box is around the first portion of the skin; select one of the four vertices of the second minimum area-bounding box as a second anchor point, wherein the selected vertex is underneath the skin and is furthest away from the mass out of the four vertices of the second minimum area-bounding box; calculate a second shortest distance from the second anchor point to the mass; and compare the first shortest distance to the second shortest distance to determine which distance is shorter, and wherein the shorter distance of the first shortest distance and the second shortest distance is the lateral margin value.

Example 18: The system of any of examples 11-17, wherein, to determine the deep margin value, the at least one non-transitory computer-readable medium storing instructions which, when executed by the one or more processors, further cause the one or more processors to: determine a lowest 30% of points belonging to the segmented mass; and calculate a distance between a point of the lowest 30% of points and an edge of the biopsy directly below the point, wherein the distance is respectively calculated for each point of the lowest 30% of points, and wherein a lowest value of the calculated distances is the deep margin value.

Example 19: The system of any of examples 11-18, wherein the GUI displays the lateral margin value with a lateral margin line, the lateral margin line indicating a lateral margin distance, and wherein the GUI displays the deep margin value with a deep margin line, the deep margin line indicating a deep margin distance.

Example 20: A non-transitory computer-readable medium, the non-transitory computer-readable medium storing instructions which, when executed by one or more processors of a computing system, cause the one or more processors to: receive an image including one or more biopsies from a database; input the image into a biopsy segmentation machine learning model; identify and segment, based on an application of the biopsy segmentation machine learning model, a biopsy of the one or more biopsies in the image to form a base image including the biopsy, wherein the biopsy includes a mass and surrounding tissue; input the base image into an ambiguity classifier machine learning model; determine, based on an application of the ambiguity classifier machine learning model, the mass in the base image is distinct from the surrounding tissue; classify, based on the application of the ambiguity classifier machine learning model, the base image as a valid image; input the valid image into a rotation classifier machine learning model; determine, based on an application of the rotation classifier machine learning model, a rotation angle associated with the valid image; rotate the valid image based on the rotation angle to form a properly oriented image, such that skin of the biopsy is at a top of the valid image; input the properly oriented image into a skin and tumor segmentation machine learning model; identify and segment, based on an application of the skin and tumor segmentation machine learning model, the skin and the mass in the properly oriented image to form a marked image; determine a lateral margin value and a deep margin value based on the segmented skin and the mass shown in the marked image; and generate computer-executable instructions configured to cause a user computing device to construct and display a graphical user interface (GUI) that presents the lateral margin value and the deep margin value in association with the marked image.

What is claimed is:

1. A computer-implemented method for automating margin measurement for mass removal comprising:
receiving, by one or more processors, an image including one or more biopsies from a database;
inputting the image, by the one or more processors, into a biopsy segmentation machine learning model;
identifying and segmenting, by the one or more processors, based on an application of the biopsy segmentation machine learning model, a biopsy of the one or more biopsies in the image to form a base image including the biopsy, wherein the biopsy includes a mass and surrounding tissue;
inputting the base image, by the one more processors, into an ambiguity classifier machine learning model;
determining, by the one or more processors, based on an application of the ambiguity classifier machine learning model, the mass in the base image is distinct from the surrounding tissue;
classifying, by the one or more processors, based on the application of the ambiguity classifier machine learning model, the base image as a valid image;
inputting the valid image, by the one or more processors, into a rotation classifier machine learning model;
determining, by the one or more processors, based on an application of the rotation classifier machine learning model, a rotation angle associated with the valid image;
rotating, by the one or more processors, the valid image based on the rotation angle to form a properly oriented image, such that skin of the biopsy is at a top of the valid image;
inputting the properly oriented image, by the one or more processors, into a skin and tumor segmentation machine learning model;
identifying and segmenting, by the one or more processors, based on an application of the skin and tumor segmentation machine learning model, the skin and the mass in the properly oriented image to form a marked image;
determining, by the one or more processors, a lateral margin value and a deep margin value based on the segmented skin and the mass shown in the marked image; and
generating, by the one or more processors, computer-executable instructions configured to cause a user computing device to construct and display a graphical user interface (GUI) that presents the lateral margin value and the deep margin value in association with the marked image.

2. The computer-implemented method of claim 1, further comprising:
predicting, by the one or more processors, based on the lateral margin value and the deep margin value, a chance of reoccurrence of the mass.

3. The computer-implemented method of claim 1, wherein the rotation angle includes zero or more 15-degree increments.

4. The computer-implemented method of claim 1, wherein the biopsy segmentation machine learning model is a transformer-based segmentation model, and wherein the skin and tumor segmentation machine learning model is a transformer-based segmentation model.

5. The computer-implemented method of claim 4, wherein the ambiguity classifier machine learning model is a residual neural network, and wherein the rotation classifier machine learning model is a scalable convolutional neural network.

6. The computer-implemented method of claim 1, wherein determining the lateral margin value comprises:
calculating a first minimum area-bounding box around the skin, the first minimum area-bounding box including four vertices;
selecting one of the four vertices of the first minimum area-bounding box as a first anchor point, wherein the selected vertex is underneath the skin and is furthest away from the mass out of the four vertices of the first minimum area-bounding box; and
calculating a first shortest distance from the first anchor point to the mass, wherein the first shortest distance is the lateral margin value.

7. The computer-implemented method of claim 6, wherein the mass separates the skin into a first portion and a second portion, and wherein determining the lateral margin value further comprises:
calculating a second minimum area-bounding box around the second portion of the skin, the second minimum area-bounding box including four vertices, wherein the first minimum area-bounding box is around the first portion of the skin;
selecting one of the four vertices of the second minimum area-bounding box as a second anchor point, wherein the selected vertex is underneath the skin and is furthest away from the mass out of the four vertices of the second minimum area-bounding box;
calculating a second shortest distance from the second anchor point to the mass; and
comparing the first shortest distance to the second shortest distance to determine which distance is shorter, and wherein the shorter distance of the first shortest distance and the second shortest distance is the lateral margin value.

8. The computer-implemented method of claim 1, wherein determining the deep margin value comprises:
determining a lowest 30% of points belonging to the segmented mass; and
calculating a distance between a point of the lowest 30% of points and an edge of the biopsy directly below the point, wherein the distance is respectively calculated for each point of the lowest 30% of points, and wherein a lowest value of the calculated distances is the deep margin value.

9. The computer-implemented method of claim 1, wherein the GUI displays the lateral margin value with a lateral margin line, the lateral margin line indicating a lateral margin distance, and wherein the GUI displays the deep margin value with a deep margin line, the deep margin line indicating a deep margin distance.

10. The computer-implemented method of claim 9, wherein the GUI displays a boundary box around the mass, and wherein the GUI displays a chance of reoccurrence of the mass based on the lateral margin value and the deep margin value.

11. A system comprising:
one or more processors of a computing system; and
at least one non-transitory computer-readable medium storing instructions which, when executed by the one or more processors, cause the one or more processors to:
receive an image including one or more biopsies from a database;
input the image into a biopsy segmentation machine learning model;
identify and segment, based on an application of the biopsy segmentation machine learning model, a biopsy of the one or more biopsies in the image to form a base image including the biopsy, wherein the biopsy includes a mass and surrounding tissue;
input the base image into an ambiguity classifier machine learning model;
determine, based on an application of the ambiguity classifier machine learning model, the mass in the base image is distinct from the surrounding tissue;
classify, based on the application of the ambiguity classifier machine learning model, the base image as a valid image;
input the valid image into a rotation classifier machine learning model;
determine, based on an application of the rotation classifier machine learning model, a rotation angle associated with the valid image;
rotate the valid image based on the rotation angle to form a properly oriented image, such that skin of the biopsy is at a top of the valid image;
input the properly oriented image into a skin and tumor segmentation machine learning model;
identify and segment, based on an application of the skin and tumor segmentation machine learning model, the skin and the mass in the properly oriented image to form a marked image;
determine a lateral margin value and a deep margin value based on the segmented skin and the mass shown in the marked image; and
generate computer-executable instructions configured to cause a user computing device to construct and display a graphical user interface (GUI) that presents the lateral margin value and the deep margin value in association with the marked image.

12. The system of claim 11, the at least one non-transitory computer-readable medium storing instructions which, when executed by the one or more processors, further cause the one or more processors to:
predict, based on the lateral margin value and the deep margin value, a chance of reoccurrence of the mass.

13. The system of claim 11, wherein the rotation angle includes zero or more 15-degree increments.

14. The system of claim 11, wherein the biopsy segmentation machine learning model is a transformer-based segmentation model, and wherein the skin and tumor segmentation machine learning model is a transformer-based segmentation model.

15. The system of claim 14, wherein the ambiguity classifier machine learning model is a residual neural network, and wherein the rotation classifier machine learning model is a scalable convolutional neural network.

16. The system of claim 11, wherein, to determine the lateral margin value, the at least one non-transitory computer-readable medium storing instructions which, when executed by the one or more processors, further cause the one or more processors to:
calculate a first minimum area-bounding box around the skin, the first minimum area-bounding box including four vertices;
select one of the four vertices of the first minimum area-bounding box as a first anchor point, wherein the selected vertex is underneath the skin and is furthest away from the mass out of the four vertices of the first minimum area-bounding box; and
calculate a first shortest distance from the first anchor point to the mass, wherein the first shortest distance is the lateral margin value.

17. The system of claim 16, wherein the mass separates the skin into a first portion and a second portion, and wherein, to determine the lateral margin value, the at least one non-transitory computer-readable medium storing instructions which, when executed by the one or more processors, further cause the one or more processors to:
calculate a second minimum area-bounding box around the second portion of the skin, the second minimum area-bounding box including four vertices, wherein the first minimum area-bounding box is around the first portion of the skin;
select one of the four vertices of the second minimum area-bounding box as a second anchor point, wherein the selected vertex is underneath the skin and is furthest away from the mass out of the four vertices of the second minimum area-bounding box;
calculate a second shortest distance from the second anchor point to the mass; and
compare the first shortest distance to the second shortest distance to determine which distance is shorter, and wherein the shorter distance of the first shortest distance and the second shortest distance is the lateral margin value.

18. The system of claim 11, wherein, to determine the deep margin value, the at least one non-transitory computer-readable medium storing instructions which, when executed by the one or more processors, further cause the one or more processors to:
determine a lowest 30% of points belonging to the segmented mass; and
calculate a distance between a point of the lowest 30% of points and an edge of the biopsy directly below the point, wherein the distance is respectively calculated for each point of the lowest 30% of points, and wherein a lowest value of the calculated distances is the deep margin value.

19. The system of claim 11, wherein the GUI displays the lateral margin value with a lateral margin line, the lateral margin line indicating a lateral margin distance, and wherein the GUI displays the deep margin value with a deep margin line, the deep margin line indicating a deep margin distance.

20. A non-transitory computer-readable medium, the non-transitory computer-readable medium storing instructions which, when executed by one or more processors of a computing system, cause the one or more processors to:
receive an image including one or more biopsies from a database;
input the image into a biopsy segmentation machine learning model;
identify and segment, based on an application of the biopsy segmentation machine learning model, a biopsy of the one or more biopsies in the image to form a base image including the biopsy, wherein the biopsy includes a mass and surrounding tissue;
input the base image into an ambiguity classifier machine learning model;
determine, based on an application of the ambiguity classifier machine learning model, the mass in the base image is distinct from the surrounding tissue;
classify, based on the application of the ambiguity classifier machine learning model, the base image as a valid image;
input the valid image into a rotation classifier machine learning model;
determine, based on an application of the rotation classifier machine learning model, a rotation angle associated with the valid image;
rotate the valid image based on the rotation angle to form a properly oriented image, such that skin of the biopsy is at a top of the valid image;
input the properly oriented image into a skin and tumor segmentation machine learning model;
identify and segment, based on an application of the skin and tumor segmentation machine learning model, the skin and the mass in the properly oriented image to form a marked image;
determine a lateral margin value and a deep margin value based on the segmented skin and the mass shown in the marked image; and
generate computer-executable instructions configured to cause a user computing device to construct and display a graphical user interface (GUI) that presents the lateral margin value and the deep margin value in association with the marked image.
